(19) United States
(12) Patent Application Publication
(10) Pub. No.: US 2005/0281871 A1
(43) Pub. Date: Dec. 22, 2005

(54) Methods for coating substrates for pharmaceutical uses with a mixture of two film-forming coating agents

(75) Inventors: Hans-Ulrich Petereit, Darmstadt (DE); Christian Meier, Darmstadt (DE); Erna Roth, Darmstadt (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Roehm Gmbh & Co. KG, Darmstadt (DE)

(21) Appl. No.: 10/529,636

(22) PCT Filed: Oct. 18, 2003
(86) PCT No.: PCT/EP03/11545

§ 371 Date: Mar. 31, 2005

(30) Foreign Application Priority Data
Dec. 20, 2002 (DE) 10260921.7

(51) Int. Cl. A61K 9/48; A61K 9/24

(52) U.S. Cl. 424/451; 424/472; 427/2.14

(57) ABSTRACT

The invention relates to methods for producing pharmaceuticals or parts of pharmaceuticals or food supplements or parts thereof, by coating substrates with a mixture of two film-forming coating agents that can contain other pharmaceutically standard additives, especially softeners and/or a pharmaceutical active ingredient.

The invention relates to a method for coating substrates for pharmaceutical uses with a mixture of two film-forming coating agents.

1. Prior art

Abletshauser C. B., describes in “Film coating of pellets with insoluble polymers obtained in situ crosslinking in fluidized bed” in Journal of Controlled Release 27 (1993), pp. 149-156, a method in which a film-forming polymer, sodium alginate, in aqueous solution and a crosslinker, e.g. a CaCl₂ solution or a (meth)acrylate copolymer with tertiary amino group radicals (EUDRAGIT E@), are sprayed simultaneously from two separate spray nozzles onto active ingredient-containing pellets. The film application can take place for example in a fluidized bed apparatus with two spray nozzles installed therein. The method has an approximately equivalent result to sequential application of the two components, but has the advantage of saving time.

WO 00/05307 describes a method for producing a coating agent and binder for oral or dermal pharmaceutical forms consisting of (a) 35-98% by weight of a copolymer consisting of free-radical polymerized C1 to C4 esters of acrylic or methacrylic acid and further (meth)-acrylate monomers which have functional tertiary ammonium groups, and (b) 1-50% by weight of a plasticizer, and 1-15% by weight of an emulsifier with an HLB of at least 14, where components (a), (b) and (c) are mixed together with or without addition of water and, where appropriate, with addition of an active pharmaceutical ingredient and further conventional additives, and the coating agent and binder is produced by melting, casting, spreading or spraying, where the copolymer (a) is introduced in powder form with an average particle size of 1-40 μm.

EP-A 0 848 960 describes an adhesive and binder for dermal or transdermal therapeutic systems consisting of (a1) 55-99.9% by weight of a (meth)acrylate copolymer of structural and functional monomers, where the functional monomers have tertiary or quaternary amino groups, (a2) 0.1-45% by weight of an acidic group-containing acrylate or (meth)acrylate polymer or copolymer and (b) 25-80% by weight, based on the total of (a1) and (a2), of a plasticizer. A transdermal therapeutic system can be produced by incorporating an active pharmaceutical ingredient by coating or by spraying or painting of solutions, dispersions, suspensions or melts of an adhesive and binder and subsequently drying or cooling.

U.S. Pat. No. 6,368,629 describes pH-dependent colonic release systems which comprise a core, an inner polymer coating, e.g. a mixture of EUDRAGIT® E and EUDRAGIT® RS, and have an outer coating of a polymer with anionic groups, e.g. a EUDRAGIT® L.

Problem and Solution

It is possible by means of the mixture, described in EP-A 0 848 960, of two (meth)acrylate copolymers to produce transdermal therapeutic systems with advantageous properties in relation to active ingredient release. It would be desirable to be able to apply this release system also to coating agents for pharmaceutical substrates such as, for example, active ingredient-containing tablet cores.

This is in principle possible without difficulty if organic solutions of the two ingredients are mixed and used for spray applications. The disadvantage of this procedure is the use of organic solvents which are known to involve problems in safety at work and in environmental protection.

If aqueous dispersions, instead of organic solutions, of the (meth)acrylate copolymers are chosen as initial basis, the difficulty arises that mixtures of the two (meth)acrylate copolymers show incompatible behavior and remain sprayable for only a short time. This means that mixed dispersions become unstable and are prone to aggregation or coagulation after only a short time. Since even slight aggregate formation lead to blockage of spray nozzles, such mixture cannot at present be employed industrially in an acceptable manner.

It is true that said aggregate formation can be avoided in aqueous systems by adding comparatively large amounts, 10% by weight or more, of nonionic emulsifiers. However, this is problematic because the aim in pharmaceutical applications is to keep the use of emulsifiers at a low level. The presence of large amounts of these substances frequently leads to problems with the long-term stability of the produced pharmaceutical forms. Thus, unwanted interactions with the active ingredient may occur during storage. Manifestation of inhomogeneity in the polymer coatings are also possible. Both these are undesired and, of course, unacceptable for pharmaceuticals.

The problem was therefore regarded as being to make (meth)acrylate copolymer mixtures like those described in EP-A 0 848 960 for transdermal systems also available for aqueous spray application systems. It is moreover intended that the use of nonionic emulsifiers either be completely avoidable or take place only in small amounts. The resulting coatings are intended to be of satisfactory quality, non-tacky and have long-term stability.

The problem is solved by a

method for producing pharmaceutical forms or parts of pharmaceutical forms or food supplements or parts thereof,

by coating substrates with a mixture of two film-forming coating agents which may comprise further pharmaceutically customary additives, especially plasticizers and/or an active pharmaceutical ingredient,

where the first film-forming coating agent

is a (meth)acrylate copolymer of 30 to 80% by weight free-radical polymerized C₁ to C₄ alkyl esters of acrylic or methacrylic acid and 70 to 20% by weight (meth)acrylate monomers having a tertiary amino group in the alkyl radical,

and the second film-forming coating agent is a polymer having anionic groups,

with the proviso that the film-forming coating agents comprise, based on the dry matter of the mixture, no or not more than 20% by weight of a plasticizer and no or not more than 5% by weight of a nonionic emulsifier,
characterized in that

the film-forming coating agents are initially separate from one another in the form of liquid, sprayable solutions or dispersions, and

are simultaneously sprayed by spray application using one or more spray devices which, singly or together, atomize liquids separately, and whose spray beams overlap,

in such a way that the incompatible individual portions are mixed in the spraying process, the mixture impinges on the substrate and forms thereon, after evaporation of the water, a film coating, resulting in the pharmaceutical form food supplement or parts thereof.

The simultaneous spraying according to the invention of the otherwise mutually incompatible components and the mixing thereof in the spray beam makes it possible to use the polymer mixture for spray application. A further advantage of this procedure is inter alia that additives such as plasticizers or nonionic emulsifiers can be kept at low levels in terms of amount or be entirely avoided.

Implementation of the Invention

The invention relates to a method for producing pharmaceutical forms or parts of pharmaceutical forms or food supplements or parts thereof, by coating substrates with a mixture of two film-forming coating agents which may comprise further pharmaceutically customary additives, especially plasticizers and/or an active pharmaceutical ingredient,

where the first film-forming coating agent is a (meth)acrylate copolymer of 30 to 80% by weight free-radical polymerized C₁ to C₄ alkyl esters of acrylic or methacrylic acid and 70 to 20% by weight (meth)acrylate monomers having a tertiary amino group in the alkyl radical,

and the second film-forming coating agent is a polymer having anionic groups,

with the proviso that the film-forming coating agents comprise, based on the dry matter of the mixture, no or not more than 20% by weight of a plasticizer and no or not more than 5% by weight of a nonionic emulsifier,
characterized in that

the film-forming coating agents are initially separate from one another in the form of liquid, sprayable solutions or dispersions, and

are simultaneously sprayed by spray application using one or more spray devices which, singly or together, have at least two separate nozzles for liquids, and whose spray beams overlap,

in such a way that the incompatible individual portions are mixed in the spraying process, the mixture impinges on the substrate and forms thereon, after evaporation of the water, a film coating, resulting in the pharmaceutical form food supplement or parts thereof.

The Film-Forming Coating Agents

The film-forming coating agents are in the form of solutions or sprayable dispersions. Each of the two coating agents may be in one or the other form. The dispersions may comprise for example a solids content of from 10 to 60, preferably 20 to 40, % by weight (meth)acrylate copolymer. The (meth)acrylate copolymers are present in the water in a fine dispersion in the form of particles with particle sizes in the range from, for example, 5 nm to 30 μm, preferably 10 nm to 500 nm. The dispersions are each stable as such. On removal of water by drying after the spraying, the particles combine and result in continuous (meth)acrylate copolymer coatings on the particular substrate.

Conventional pharmaceutical excipients may additionally be present, but with the proviso that the film-forming coating agents comprise, based on the dry matter of the mixture, no or not more than 20% by weight of a plasticizer and no or not more than 5% by weight of a nonionic emulsifier.

The film-forming coating agents (dispersions) comprise, in total, based on the dry matter of the mixture, no or not more than 20% by weight of a plasticizer and no or not more than 5% by weight of a nonionic emulsifier.

The First Film-Forming Coating Agent

The (meth)acrylate copolymer is composed of 30 to 80% by weight of free-radical polymerized C₁ to C₄ alkyl esters of acrylic or methacrylic acid and 70 to 20% by weight of (meth)acrylate monomers with a tertiary amino group in the alkyl radical.

Suitable monomers with functional tertiary amino groups are listed in U.S. Pat. No. 4,705,695, column 3, line 64 to column 4, line 13. Particular mention should be made of dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate and diethylamino-2,2-dimethyl)propyl methacrylate. Dimethylaminoethyl methacrylate is particularly preferred.

The content of monomers with tertiary amino groups in the copolymer can advantageously be between 20 and 70% by weight, preferably between 40 and 60% by weight. The proportions of C₁ to C₄ alkyl esters of acrylic or methacrylic acid is 70-30% by weight. Mention should be made of methyl methacrylate, ethyl methacrylate, butyl methacrylate, methyl acrylate, ethyl acrylate and butyl acrylate.

A suitable (meth)acrylate copolymer with tertiary amino groups may be composed for example of 20-30% by weight methyl methacrylate, 20-30% by weight butyl methacrylate and 60-40% by weight dimethylaminoethyl methacrylate.

A specifically suitable commercially available (meth)acrylate copolymer with tertiary amino groups is composed for example of 25% by weight methyl methacrylate, 25% by weight butyl methacrylate and 50% by weight dimethylaminoethyl methacrylate (EUDRAGIT® E100).

The (meth)acrylate copolymer can be obtained in a manner known per se by free-radical bulk, solution, bead or emulsion polymerization. It may before processing be brought to the suitable particle size range by suitable grinding, drying or spraying processes.

Suitable apparatuses for producing powders are familiar to the skilled worker, e.g. air jet mills, pinned disk mills, compartment mills. It is possible where appropriate to include appropriate sieving steps. A suitable mill for industrial large quantities is, for example, an opposed jet mill (Multi No. 4200) which is operated with a gage pressure of about 6 bar.

The average particle size of the powders can be determined as follows:

By air jet sieving to divide up the ground product easily into a few fractions. This method is somewhat less exact than the alternatives in this range of measurement.

A further very suitable measurement method is laser diffraction to determine the particle size distribution. Commercially available apparatuses permit measurement in air (Malvern S3.01 particle sizer) or preferably in liquid media (LOT, Galai CIS 1). A precondition for measurement in liquids is that the polymer does not dissolve therein or the particles change in another way during the measurement. A suitable medium is, for example, a highly dilute (approx. 0.02% strength) aqueous polysorbate 80 solution.

At least 70, preferably 90, % of the particles based on the mass (mass distribution) can preferably be in the 1-40 μm size range.

(Meth)acrylate copolymers with an average particle diameter must be in the range between 1 and 40, preferably between 5 and 35, in particular between 10 and 20, μm are preferred. (EUDRAGIT® EPO type).

The Second Film-Forming Coating Agent

The second film-forming coating agent is a polymer having anionic groups and may be a cellulose derivative, e.g. cellulose acetate phthalate (CAP), cellulose acetate succinate (CAS), cellulose acetate trimelitate (CAT), hydroxypropylmethylcellulose phthalate (HPMCP), a polyvinyl acetate derivative, e.g. polyvinyl acetate phthalate, (PVAP) or a (meth)acrylate copolymer.

The second film-forming coating agent is preferably a (meth)acrylate copolymer of 40 to 95% by weight free-radical polymerized C₁ to C₄ alkyl esters of acrylic or methacrylic acid and comprises 5 to 60% by weight (meth)acrylate monomers having an anionic group in the alkyl radical.

The (meth)acrylate copolymer consists of 40 to 100, preferably 45 to 99, in particular 85 to 95, % by weight of free-radical polymerized C₁ to C₄ alkyl esters of acrylic or methacrylic acid and may comprise 0 to 60, preferably 1 to 55, in particular 5 to 15, % by weight of (meth)acrylate monomers having an anionic group in the alkyl radical.

Normally, the proportions mentioned add up to 100% by weight. However, small amounts in the range from 0 to 10, e.g. 1 to 5, % by weight of further vinylically copolymerizable monomers such as, for example, hydroxyethyl methacrylate or hydroxyethyl acrylate may additionally be present without this leading to an impairment or alteration of the essential properties.

C₁ to C₄ alkyl esters of acrylic or methacrylic acid are in particular methyl methacrylate, ethyl methacrylate, butyl methacrylate, methyl acrylate, ethyl acrylate and butyl acrylate.

A (meth)acrylate monomer having an anionic group in the alkyl radical may be for example acrylic acid, but preferably methacrylic acid.

Also suitable are anionic (meth)acrylate copolymers composed of 40 to 60% by weight methacrylic acid and 60 to 40% by weight methyl methacrylate or 60 to 40% by weight ethyl acrylate (EUDRAGIT® L or EUDRAGIT® L100-55 types).

EUDRAGIT® L100-55 is a copolymer of 50% by weight ethyl acrylate and 50% by weight methacrylic acid. EUDRAGIT® L30-55 is a dispersion comprising 30% by weight EUDRAGIT® L 100-55.

Likewise suitable are anionic (meth)acrylate copolymers of 20 to 40% by weight methacrylic acid and 80 to 60% by weight methyl methacrylate (EUDRAGIT® S type).

(Meth)acrylate copolymers consisting of 10 to 30% by weight methyl methacrylate, 50 to 70% by weight methyl acrylate and 5 to 15% by weight methacrylic acid (EUDRAGIT® FS type) are particularly well suited. EUDRAGIT® FS is a copolymer of 25% by weight methyl methacrylate, 65% by weight methyl acrylate and 10% by weight methacrylic acid. EUDRAGIT® FS 30 D is a dispersion comprising 30% by weight EUDRAGIT® FS.

The copolymers are obtained in a manner known per se by free-radical bulk, solution, bead or emulsion polymerization. They must before processing be brought to the particle size range of the invention by suitable grinding, drying or spraying processes. This can take place by simple crushing of extruded and cooled pellets or hot cut.

The use of powders may be advantageous especially on mixture with other powders or liquids. Suitable apparatuses for producing powders are familiar to the skilled worker, e.g. air jet mills, pinned disk mills, compartment mills. It is possible where appropriate to include appropriate sieving steps. A suitable mill for industrial large quantities is, for example, an opposed jet mill (Multi No. 4200) which is operated with a gage pressure of about 6 bar.

The film-forming polymers are in each case in the form of a solution or aqueous disperse system which permits film formation under the usual conditions of pharmaceutical coating methods.

Further commercially available anionic polymers:

cellulose glycolate (Duodcell®)

cellulose acetate phthalate (CAP, Cellulosi acetas,

PhEur, Cellulose acetate phthalate, NF, Aquateric®)

cellulose acetate succinate (CAS)

cellulose acetate trimeliates (CAT)

hydroxypropylmethylcellulose phthalate (HPMCP, HP 50, HP 55)

polyvinyl acetate phthalate (PVAP)

vinyl acetate-vinylpyrolidone copolymer (PVAc, Kollidon® VA64)

Substrates

The substrates for pharmaceutical applications may be for example active ingredient crystals, active ingredient-containing cores, granules, tablets, pellets or capsules. These may be of regular or irregular shape.

The size of granules, pellets or crystals is between 0.01 and 2.5 mm, that of tablets is between 2.5 and 30.0 mm. Capsules consist for example of gelatin, starch or cellulose derivatives.

The substrates may comprise a biologically active substance (active ingredient) up to 95% and further pharmaceutical excipients up to 99.9% by weight.

Usual production processes are direct compression, compression of dry, moist or sintered granules, extrusion and subsequent rounding off, wet or dry granulation or direct pelleting (e.g. on plates) or by binding of powders (powder layering) onto active ingredient-free beads (nonpareilles) or active ingredient-containing particles.

Besides the active ingredient, further pharmaceutical excipients may be present, such as, for example, binders such as cellulose and derivatives thereof, polyvinylpyrrolidone (PVP), humectants, disintegration promoters, lubricants, disintegrants, (meth)acrylates, starch and derivatives thereof, sugar solubilizers or others.

Spray Device

It is possible to employ or use as spray device those having two or more two-fluid nozzles or one or more three-fluid nozzles.

In a two-fluid nozzle or a three-fluid nozzle, in each case one of the nozzle orifices is supplied with compressed air to atomize the liquid which is sprayed at the same time. The other or the two other spray nozzles serve to eject the respective film-forming coating agent. To carry out the method, therefore, either at least two two-fluid nozzles are required, where one in each case sprays the first film-forming coating agent and the liquid with the further substance, or a three-fluid nozzle, which sprays both simultaneously, is required.

The delivery rates of the sprayed liquids can be influenced independently of one another by the setting of parameters such as, for example, the pump outputs or the spraying pressure and/or the air delivery rates. It is possible in principle for the settings of the spray devices to be carried out manually during the spraying process. In order to obtain reproducible results, it is preferred to control the parameters which influence the delivery rates of the sprayed liquids by means of fixed programs, e.g. by electronic means.

Examples of commercially available spray devices are, for example, the Pilot SIL XII spray gun (double two-fluid nozzle; manufactured by Walther, Wuppertal, Germany), the “Concentric Dual-Feed Nozzle” model (three-fluid nozzle, manufactured by ShinEtsu, Japan) or model 946-S15 (three-fluid nozzle, manufactured by Dusen Schlick GmbH, D-96253 Untersiemau, Germany).

Spray Application

Spray application takes place by means of one or more spray devices which have, singly or together, at least two separate nozzles for liquids and whose spray beams overlap.

The two film-forming coating agents are initially separate from one another in the form of sprayable dispersions and are sprayed simultaneously in such a way that the incompatible individual portions mix during the spraying, impinge on the substrate and thereon, after evaporation of the water content, a uniform film coating.

The spray solutions are fed to the nozzles through tubing by means of pumps which generate low shear forces. Tubing pumps are preferred.

In order to ensure good mixing, the simultaneous spraying preferably takes place with a respective spraying pressure in the range from 0.8 to 1.5 bar.

The film-forming coating agents are preferably employed in a mixing ratio of from 9:1 to 1:9 based on the total polymer mass of the film coating.

The spray application can take place for example in a drum coater, a coating pan, a fluidized bed apparatus or a spray sifter.

The spray application can take place using manually guided spray devices. However, better and more reproducible results are usually obtained with spray devices which are fixed installations, so that these are preferred.

Equipment

The method is particularly preferably carried out with drum coaters, coating pans, fluidized bed apparatuses or spray sifters comprising as spray device one or more three-fluid nozzles, in particular as fixed installation.

Coated Pharmaceutical Form or Coated Food Supplement

Coated pharmaceutical forms or parts of pharmaceutical forms or food supplements or parts thereof can in particular be produced or obtained by means of the method of the invention. The sprayed individual portions are mixed together within fractions of seconds during the spray application and, through the evaporation of the water which proceeds virtually simultaneously, form a polymer matrix on the surface of the substrates. The resulting molecular matrix structure should therefore differ from a matrix structure produced when both film-forming coating agents is present in a polymer dispersion before the spraying. Despite this difference, no adverse effects compared with conventional methods are found in the quality of the coating, e.g. gloss or uniformity; on the contrary, novel properties differing from the initial polymers are obtained. It is surprising that slow-release pharmaceutical forms which have pH-independent release and display partially sigmoidal release profiles are obtained.

The applied amount of polymer depends on the shape and size of the substrate. A complete coating is always necessary for reliable control of release. This amount amount of polymer is above 1% by weight for tablets and above 5% by weight for granules, powders or pellets, in each case based on the uncoated substrate.

The air pressure generating the spray mist is between 0.5 and 3 bar, preferably between 1 and 2 bar. Only in rare cases where the viscosity of one or both spray liquids is distinctly higher than water may it be necessary to increase the spraying pressure further.

The spraying rate of the two individual components may differ and depends greatly on the batch size, the individual formula and the drying capacity, determined by the air throughput, of the equipment used. Ordinarily, the total of the spraying rates of the two liquids is 1 to 15 g/kg of cores×min, preferably 5 to 10 g/kg of cores×min).

The product temperature to be maintained during the spraying depends on the formula of the individual components used and the properties, determined thereby, of the film former. Guideline values are from 15 to 50° C., preferably 20 to 40° C., particularly preferably 25 to 35° C.

It is also possible where appropriate to apply a rapidly released initial dose. The active ingredient is in this case incorporated into a water-soluble binder.

The pharmaceutical form may comprise an active ingredient from the class of analgesics, antiallergics, antiarrhythmics, antibiotics, chemotherapeutics, antidiabetics, antidotes, antiepileptics, antihypertensives, antihypotensives, anticoagulants, antimycotics, antiinflammatory agents, beta-receptor blockers, calcium antagonists and ACE inhibitors, bronchospasmolytics/antiasthmatics, cholinergics, corticoids (for internal use), diuretics, enzyme inhibitors, enzyme preparations and transport proteins, expectorants, geriatrics, gout remedies, influenza remedies, hormones and their inhibitors, hypnotics/sedatives, cardiac drugs, lipid-lowering agents, parathyroid hormones/calcium metabolism regulators, psychoactive drugs, sex hormones and their inhibitors, spasmolytics, sympatholytics, sympathomimetics, vitamins, wound-treatment agents, cytostatics, nucleic acids, proteins or peptides.

Medicinal substances in use can be found in reference works such as, for example, the Rote Liste or the Merck Index.

Biologically Active Substances:

The medicinal substances employed for the purposes of the invention are intended to be used on or in the human or animal body in order,
1. to cure, to alleviate, to prevent or to diagnose disorders, conditions, physical damage or pathological symptoms.
2. to reveal the condition, the status or the functions of the body or mental states.
3. to replace active substances or body fluids produced by the human or animal body.
4. to ward off, to eliminate or to render harmless pathogens, parasites or exogenous substances, or
5. to influence the condition, the status or the functions of the body or mental states.

The formulation of the invention is suitable for administration of in principle any active pharmaceutical ingredients or biologically active substances which can preferably be administered in slow-release form.

These pharmaceutically active substances may belong to one or more active ingredient classes such as ACE inhibitors, adrenergics, adrenocorticosteroids, acne therapeutic agents, aldose reductase inhibitors, aldosterone antagonists, alpha-glucosidase inhibitors, alpha 1 antagonists, remedies for alcohol abuse, amino acids, amebicides, anabolics, analeptics, anesthetic additions, anesthetics (non-inhalational), anesthetics (local), analgesics, androgens, angina therapeutic agents, antagonists, antiallergics, antiallergics such as PDE inhibitors, antiallergics for asthma treatment, further antiallergics (e.g. leukotriene antagonists, antianemics, antiandrogens, antianxiolytics, antiarthritics, antiarrhythmics, antiatheriosclerotics, antibiotics, anticholinergics, anticonvulsants, anti-depressants, antidiabetics, antidiarrheals, antidiuretics, antidotes, antiemetics, antiepileptics, antifibrinolytics, antiepileptics, antihelmintics, antihistamines, antihypotensives, antihypertensives, antihypertensives, antihypotensives, anticoagulants, antimycotics, antiestrogens, antiestrogens (non-steroidal), antiparkinson agents, antiinflammatory agents, antiproliferative active ingredients, antiprotozoal active ingredients, antirheumatics, antischistosomicides, antispasmolytics, antithrombotics, antitussives, appetite suppressants, arteriosclerosis remedies, bacteriostatics, beta-blockers, beta-receptor blockers, bronchodilators, carbonic anhydrase inhibitors, chemotherapeutic agents, choleretics, cholinergics, cholinergic agonists, cholinesterase inhibitors, agents for the treatment of ulcerative colitis, diuretics, ectoparasiticides, emetics, enzymes, enzyme inhibitors, enzyme inhibitors, active ingredients to counter vomiting, fibrinolytics, fungistatics, gout remedies, glaucoma therapeutic agents, glucocorticoids, glucocorticosteroids, hemostatics, cardiac glycosides, histamine H2 antagonists, hormones and their inhibitors, immunotherapeutic agents, cardiotonics, coccidiostats, laxatives, lipid-lowering agents, gastrointestinal therapeutic agents, malaria therapeutic agents, migraine remedies, microbiocides, Crohn's disease, metastasis inhibitors, migraine remedies, mineral preparations, motility-increasing active ingredients, muscle relaxants, neuroleptics, active ingredients for treatment of estrogens, osteoporosis, otologicals, antiparkinson agents, phytopharmaceuticals, proton pump inhibitors, prostaglandins, active ingredients for treating benign prostate hyperblasia, active ingredients for treating pruritus, psoriasis active ingredients, psychoactive drugs, radical scavengers, renin antagonists, thyroid therapeutic agents, active ingredients for treating seborrhea, active ingredients to counter seasickness, spasmolytics, alpha- and beta-sympathomimetics, platelet aggregation inhibitors, tranquilizers, ulcer therapeutic agents, further ulcer therapeutic agents, agents for the treatment of urolithiasis, virustatics, virustatics, vitamins, cytokines, active ingredients for combination therapy with cytostatics, cytostatics.

Examples of suitable active ingredients are acarbose, acetylsalicylic acid, aclarubicin, acyclovir, cisplatin, actinomycin, adenosylmethionine, adrenaline and adrenaline derivatives, alemtuzumab, allopurinol, almotriptan, alosetron, alprostadil, amantadine, ambroxol, amlodipine, amoxicillin, 5-aminosalicylic acid, amitriptyline, amlodipine, amoxicillin, anastrozole, androgen and androgen derivatives, atenolol, atorvastatin, azathioprine, azelaic acid, barbituric acid derivatives, balsalazide, beclomethasone, benzodiazepines, betahistine, bezafibrate, bicalutamide, bimatoprost, budesonide, bufexamac, buprenorphine, bupropion, butizine, calcium antagonists, calcium salts, candesartan, capecitabine, captopril, carbamazepine, caspofungin, cefadroxil, cefalosporins, cefditoren, cefprozil, celetoxib, cetirizine, chenodeoxycholic acid, ciclosporin, cimetidine, clarithromycin, clavulanic acid, clindamycin, clobutinol, clonidine, codeine, caffeine, colestyramine, cromoglicic acid, cotrimoxazole, coumarin and coumarin derivatives, cysteine, cytarabine, cyclophosphamide, cyproterone, cytarabine, dapiprazole, desipramine, desogestrel, desonide, disoproxil, diazepam and diazepam derivatives, dihydralazine, diltiazem, dimenhydrinate, dimethyl sulfoxide, dimeticone, dipyridarnoi, domperidone, and domperidane derivatives, donepzil, dopamine, doxazosin, doxorubizin, doxylamine, diclofenac, divalproex, drospirenone, econazole, emtricitabine, enalapril, ephedrine, epinephrine, epoetin and epoetin derivatives, eprosartan, esomeprazole, estrogen and estrogen derivatives, ethenzamide, ethinestradiol, etofenamate, etofibrate, etofylline, etonorgestrel, etoposide, famciclovir, famotidine, felodipine, fenofibrate, fentanyl, fenticonazole, fexofenadine, fluconazole, fludarabine, flunarizine, fluorouracil, fluoxetine, flurbiprofen, flupirtine, flutamide, fluvastatin, follitropin, formoterol, fosfomicin, frovatriptan, furosemide, fusidic acid, galantamine, gallopamil, ganciclovir, gemfibrozil, gentamicin, progestogen and progestogen derivatives, ginkgo, glibenclamide, glucagon, glucitol and glucitol derivatives, glucosamine and glucosamine derivatives, glycoside antibiotics, urea derivatives as oral antidiabetics, glutathione, glycerol and glycerol derivatives, hypothalamus hormones, goserelin, gyrase inhibitors, guanethidine, gyrase inhibitors, halofantrine, haloperidol, heparin and heparin derivatives, cardiac glycosides, hyaluronic acid, hydralazine, hydrochlorothiazide and hydrochlorothiazide derivatives, hydroxyomeprazole, hydroxyzine, ibuprofen, idarubicin, ifosfamide, imatinib, imipramine, indometacin, indoramine, insulin, interferons, irinotecan, isoconazole, isoprenaline, itraconazole, ivabradines, iodine and iodine derivatives, St. John's wort, potassium salts, ketoconazole, ketoprofen, ketotifen, lacidipine, lansoprazole, letrozol, levodopa, levomethadone, lipoic acid and lipoic acid derivatives, lisinopril, lisuride, lofepramine, lomustine, loperamide, loratadine, magnesium salts, macrolide antibiotics, maprotiline, mebehdazole, mebeverine, meclozine, mefenamic acid, mefloquine, meloxicam, mepindolol, meprobamate, meropenem, mesalazine, mesuximide, metamizole, metformin, methadone, methotrexate, methylnaloxone, methylnaltrexones, methylphenidate, methylprednisolone, metixen, metoclopramide, metoprolol, metronidazole, mianserin, miconazole, minocycline, minoxidil, misoprostol, mitomycin, mizolastine, modafinil, moexipril, morphinans, morphine and morphine derivatives, ergot alkaloids, nalbuphine, naloxone, naproxen, narcotine, natamycin, neostigmine, neramexan, nicergoline, nicethamide, nifedipine, niflumic acid, nimodipine, nimorazole, nimustine, nesiritide, nisoldipine, norfloxacin, novamine sulfone, noscapine, nystatin, ofloxacin, olanzapine, olsalazine, omeprazole, omoconazole, ondansetron, orlistat, oseltamivir, oxaceprol, oxacillin, oxiconazole, oxymetazoline, pantoprazole, paracetamol, paroxetine, peginterferon, penciclovir, oral penicillins, pentazocine, pentifylline, pentoxifylline, peptide antibiotics, perindopril, perphenazine, pethidine, plant extracts, phenazone, pheniramine, phenytoin, phenothiazines, phenylbutazone, phenytoin, pimozide, pindolol, piperazine, piracetam, pirenzepine, piribedil, piroxicam, pramipexol, pravastatin, prazosin, procaine, promazine, propiverine, propranolol, propyphenazone, prostaglandins, protionamide, proxyphylline, quetiapine, quinapril, quinaprilate, ramipril, ranitidine, ranolazines, reproterol, reserpine, ribavirin, rifampicin, riluzoles, risedronate, risperidone, ritonavir, ropinirol, rosiglitazone, roxatidine, roxithromycin, ruscogenin, rosuvastatin, rutoside and rutoside derivatives, sabadilla, salbutamol, salicylates, salmeterol, thyroid hormones, scopolamine, selegiline, sertaconazole, sertindole, sertralion, sildenafil, silicates, simvastatin, sitosterol, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiam, sumatriptan, suxamethonium chloride, tacrine, tacrolimus, tadalafil, taliolol, talsaclidine, tamoxifen, tazarotene, tegaserod, temazepam, teniposide, tenofovir, tenoxicam, terazosin, terbinafine, terbutaline, terfenadine, terlipressin, tertatoloL, testosterone and testosterone derivatives, tetracyclines, tetryzoline, theobromine, theophylline, theophylline derivatives, trypsins, thiamazole, thiotepa, tiagabine, tiapride, propionic acid derivatives, ticlopidine, tilidine, timolol, tinidazole, tioconazole, tioguanine, tioxolone, tiropramide, tizanidine, tolazoline, tolbutamide, tolcapone, tolnaftate, tolperisone, topiramate, topotecan, torasemide, tramadol, tramazoline, trandolapril, tranylcypromine, trapidil, trazodone, triamcinolone and triamcinolone derivatives, triamterene, trifluperidol, trifluridine, trimetazidines, trimethoprim, trimipramine, tripelennamine, triprolidine, trifosfamide, tromantadine, trometamol, tropalpine, troxerutin, tulobuterol, tyramine, tyrothricin, urapidil, ursodeoxycholic acid, theophylline ursodeoxycholic acid, valaciclovir, valdecoxib, valganciclovir, valproic acid, vancomycin, vardenafil, vecuronium chloride, venlafaxine, verapamil, vidarabine, vigabatrine, viloxazine, vinblastine, vincamine, vincristine, vindesine, vinorelbine, vinpocetine, viquidil, vitamin D and derivatives of vitamin D, warfarin, xantinol nicotinate, xipamide, zafirlukast, zalcitabine, zanamivir, zidovudine, ziprasidone, zoledronic acid, zolmitriptan, zolpidem, zoplicone, zotepine and the like.

The active ingredients can if desired also be used in the form of their pharmaceutically acceptable salts or derivatives, and in the case of chiral active ingredients it is possible to employ both optically active isomers and racemates or mixtures of diastereoisomers. If desired, the compositions of the invention may also comprise two or more active pharmaceutical ingredients.

The pharmaceutical forms are preferably multi-particulate, for example in the form of capsules, sachets, powders for reconstitution or disintegrating tablets.

Excipients Customary in Pharmacy

The film-forming coating agents should comprise, based on the dry matter of the mixture, no or not more than 20% by weight of a plasticizer and no or not more than 5% by weight of a nonionic emulsifier.

Plasticizers: Substances suitable as plasticizers ordinarily have a molecular weight between 100 and 20 000 and contain one or more hydrophilic groups in the molecule, e.g. hydroxyl, ester or amino groups. Citrates, phthalates, sebacates, castor oil are suitable. Examples of suitable plasticizers are alkyl citrates, propylene glycol, glycerol esters, alkyl phthalates, alkyl sebacates, sucrose esters, sorbitan esters, diethyl sebacate, dibutyl sebacate and polyethylene glycols 4000 to 20 000. Preferred plasticizers are tributyl citrate, triethyl citrate, acetyl triethyl citrate, dibutyl sebacate and diethyl sebacate. The amounts used are between 1 and 20, preferably 2 to 10, % % by weight based on the (meth)acrylate copolymer.

Emulsifiers

If emulsifiers are present in the coating agents, they should be toxicologically acceptable. In principle, nonionic emulsifiers are preferred for pharmaceuticals.

Suitable classes of emulsifiers are ethoxylated fatty acid esters or ethers, ethoxylated sorbitan ethers, ethoxylated alkylphenols, glycerol esters or sugar esters or wax derivatives.

Examples of suitable emulsifiers are polyoxyethylene glycerol monolaurate, polyoxyethylene glycerol monostearate, polyoxyethylene 25 cetylstearate, polyoxyethylene 25 oxypropylene monostearate, polyoxyethylene 20 sorbitan monopalmitate, polyoxyethylene 16 tert-octylphenol, polyoxyethylene 20 cetyl ether, polyethylene glycol (1000) monocetyl ether, ethoxylated castor oil, polyoxyethylene sorbitol wool wax derivatives, polyoxyethylene (25) propylene glycol stearate, polyoxyethylene sorbitol esters polyoxyethylene 25 cetylstearate, polyoxyethylene 20 sorbitan monopalmitate, polyoxyethylene 16 tertoctylphenol and polyoxyethylene 20 cetyl ether.

Dryers (non-stick agents): Dryers have the following properties: they have large specific surface areas, are chemically inert, are free-flowing and comprise fine particles. Because of these properties, they can advantageously be dispersed homogeneously in melts and reduce the tack of polymers containing highly polar comonomers as functional groups.

Examples of dryers are:

Alumina, magnesium oxide, kaolin, talc, silica (Aerosils), barium sulfate, carbon black and cellulose.

Release Agents (Mold Release Agents)

Examples of release agents are:

esters of fatty acids or fatty amides, aliphatic, long-chain carboxylic acids, fatty alcohols and esters thereof, montan waxes or paraffin waxes and metal soaps; particular mention should be made of glycerol monostearate, stearyl alcohol, glycerol behenic acid ester, cetyl alcohol, palmitic acid, canauba wax, beeswax etc.

Further excipients: Mention should be made here of, for example, stabilizers, colorants, antioxidants, wetting agents, pigments, gloss agents etc. They are used in particular as processing aids and are intended can be to ensure a reliable and reproducible production process and good long-term storage stability. Further excipients customary in pharmacy may be present in amounts of from 0.001% by weight to 30% by weight, preferably 0.1 to 10% by weight, based on the copolymer.

Preferred active ingredients are:

Morphine and its derivatives, tramadol, acetylsalicylic acid, diclofenac, indometacin, lonazolac, ibuprofen, ketoprofen, propyphenazone, naproxen, paracetamol, flurbiprofen, dimetindene, quinidine, metoprolol, propranolol, oxprenolol, pindolol, atenolol, metoprolol, disopyramide, verapamil, diltiazem, gallopamil, nifedipine, nicardipine, nisoldipine, nimodipine, amlodipine, theophylline, salbutamol, terbutaline, ambroxol, aminophylline, choline theophyllinate, pyridostigmine, piretanide, furosemide, pentoxyfylline, naftidrofuryl, buflomedil, xantinol nicotinate, bencyclane, allopurinol, norephedrine, clorphenamine isosorbide mononitrate, isosorbide dinitrate, glycerol trinitrate, molsidomine, bezafibrate, fenofibrate, gemfibrozil, cerivastatin, pravastatin, fluvastatin, lovastatin, atorvastatin, simvastatin, xantinol, metoclopramide, amitriptyline, dibenzepine, venlafaxine, thioridazine, oxazepam, lithium, nitrofurantoin, plant dry extract, ascorbic acid and potassium and the pharmaceutically used salts thereof.

### EXAMPLES

### Example 1

1.1 Production of the Cationic Spray Suspension (First Film-Forming Coating Agent):

114.0 g of EUDRAGIT® E PO (copolymer of methyl methacrylate, butyl methacrylate, and dimethylamino-ethyl methacrylate in the ratio 25:25:50 with an average particle size of 15 μm), 8.0 g of sodium lauryl sulfate, 17.1 g of dibutyl sebacate, 693.2 g of water and magnesium stearate 34.2 g are converted into a polymer dispersion by simple stirring at room temperature.

Production of the Anionic Spray Dispersion (Second Film-Forming Coating Agent):

114.0 g of talc are dispersed in 836.0 g of water with a homogenizer (Ultra Turrax) and stirred into 760.0 g of EUDRAGIT® L 30 D-55 (copolymer of 50% by weight ethyl acrylate and 50% by weight methacrylic acid).

A three-fluid nozzle, e.g. Walther Pilot SIL XII, with which the EUDRAGIT® E PO dispersion and the EUDRAGIT® L 30 D55 dispersion (suspension) are fed in separately and mixed immediately after the nozzle outlet, can be used to spray the formula described above onto 3 kg of tablets (diameter 10 mm) in a conventional coating pan at a tablet bed temperature of about 30-45° C.° C. with a spraying pressure of about 1.2 bar within 170 min to give a homogeneous film. Subsequent drying for 15 minutes results in smooth and glossy films which do not dissolve in water.

### Example 2

Production of the Cationic Spray Suspension (First Film-Forming Coating Agent):

114.0 g of EUDRAGIT® E PO, 1.14 g of sodium lauryl sulfate, 17.1 g of dibutyl sebacate, 651.8 g of water and magnesium stearate 34.2 g are converted into a polymer dispersion by simple stirring at room temperature.

Production of the Anionic Spray Dispersion:

57.0 g of talc and 17.1 g of triethyl citrate are dispersed in 486.4 g of water with a homogenizer (Ultra Turrax) and stirred into 380.0 g of EUDRAGIT® L 30 D-55.

A three-fluid nozzle, e.g. Walther Pilot SIL XII, with which the EUDRAGIT® E PO dispersion and the EUDRAGIT® L 30 D55 suspension are fed in separately and mixed immediately after the nozzle outlet, can be used to spray the formula described above onto 3 kg of tablets (diameter 10 mm) in a conventional coating pan at a tablet bed temperature of about 33-41° C. with a spraying pressure of about 1.2 bar within 117 min to give a homogeneous film. Subsequent drying for 15 minutes results in smooth and glossy films which do not dissolve in water.

### Example 3

### Release Investigations on Tablets From Example 1

An approximately 300 mg coated quinidine sulfate tablet with 5% active ingredient content is put into a paddle apparatus with 700 ml of 0.1N hydrochloric acid, 37° C. and 100 rpm, and the release of active ingredient is tested over 2 hours in this medium via a photometric absorption at the wavelength of 250.0 nm after 10, 20, 30, 60, 90 and 120 min. After 120 min in 0.1N HCl, the pH is adjusted to 6.8 with 200 ml of 0.2N Na₃PO₄. The release investigation then likewise takes place via photometric determination at the wavelengths of 234 nm after 135, 150, 180, 210, 240, 300 and 360 min. This is followed by homogenization and standardization of the total active ingredient concentration to this value as 100% value.

[image]

### Example 4

### Release Investigations on Tablets From Example 2

An approximately 300 mg coated quinidine sulfate tablet with 5% active ingredient content is put into a paddle apparatus with 700 ml of 0.1N hydrochloric acid, 37° C. and 100 rpm, and the release of active ingredient is tested over 2 hours in this medium via a photometric absorption at the wavelength of 250.0 nm after 10, 20, 30, 60, 90 and 120 min. After 120 min in 0.1N HCl, the pH is adjusted to 6.8 with 200 ml of 0.2N Na₃PO₄. The release investigation then likewise takes place via photometric determination at the wavelengths of 234 nm after 135, 150, 180, 210, 240, 300 and 360 min. This is followed by homogenization and standardization of the total active ingredient concentration to this value as 100% value.

[image]

### Example 5

A film-forming dispersion is produced from 114.0 g of EUDRAGIT® E PO, 1.14 g of sodium lauryl sulfate and 651.8 g of water by stirring at room temperature.

(Cationic Polymer Dispersion).

A fine-particle suspension is produced from 17.1 g of triethyl citrate, 57.0 g of talc and 486.4 g of water at room temperature using a homogenizer (Ultra Turrax), introduced into 380.0 g of EUDRAGIT® L 30 D 55 and mixed by simple stirring (anionic polymer dispersion). The two liquids are fed via separate tubing pumps to the nozzle heads of a multi-fluid nozzle (e.g. Walther Pilot SIL XII, and atomized so that the mists of the dispersions mix immediately after the nozzle outlet. The coating process is carried out on 3 kg of placebo tablets (diameter 10 mm) in a conventional coating pan (35 cm diameter) while feeding in hot air. The tablet bed temperature is kept at about 33-41° C. The spraying pressure of both heads was adjusted to about 1.2 bar. The spraying process lasted about 117 min. Subsequent drying for 15 minutes results in smooth, glossy pigmented films which do not dissolve in water.

### Example 6

### Comparative Example

A film-forming dispersion is produced from 114.0 g of EUDRAGIT® E PO, 1.14 g of sodium lauryl sulfate and 651.8 g of water by stirring at room temperature.

(Cationic Polymer Dispersion).

A fine-particle suspension is produced from 17.1 g of triethyl citrate, 57.0 g of talc and 486.4 g water at room temperature using a homogenizer (Ultra Turrax), introduced into 380.0 g of EUDRAGIT® L 30 D 55 and mixed by simple stirring (anionic polymer dispersion). The two suspensions are fed from separate vessels via tubing pumps to a modified two-fluid NBA 1 spray gun (from Walther Trowal) so that mixing of the two suspensions takes place inside the spray gun, i.e. shortly before the spraying nozzle. Spray application is not possible because of coagulation in the spray gun.

1. A method for producing pharmaceutical forms or parts of pharmaceutical forms or food supplements or parts thereof, comprising
coating substrates with a mixture of two film-forming coating agents which may comprise further pharmaceutically customary additives, especially plasticizers and/or an active pharmaceutical ingredient,
where the first film-forming coating agent
is a (meth)acrylate copolymer of 30 to 80% by weight free-radical polymerized C₁ to C₄ alkyl esters of acrylic or methacrylic acid and 70 to 20% by weight (meth)acrylate monomers having a tertiary amino group in the alkyl radical,
and the second film-forming coating agent
is a polymer having anionic groups,
with the proviso that the film-forming coating agents comprise, based on the dry matter of the mixture, no or not more than 20% by weight of a plasticizer and no or not more than 5% by weight of a nonionic emulsifier, wherein
the film-forming coating agents are initially separate from one another in the form of liquid, sprayable solutions or dispersions, and
are simultaneously sprayed by spray application using one or more spray devices which, singly or together, atomize liquids separately, and whose spray beams overlap,
in such a way that the incompatible individual portions are mixed in the spraying process, the mixture impinges on the substrate and forms thereon, after evaporation of the water, a film coating, resulting in the pharmaceutical form food supplement or parts thereof.

2. The method as claimed in claim 1, wherein the substrates are active ingredient crystals, active ingredient-containing cores, cores coated with an active ingredient-containing binder, tablets, granules, pellets or capsules.

3. The method as claimed in claim 1 wherein the first film-forming coating agent is a copolymer of 25% by weight methyl methacrylate, 25% by weight butyl methacrylate and 50% by weight dimethylaminoethyl methacrylate.

4. The method as claimed in claim 1 wherein the second film-forming coating agent is a polymer having anionic groups which is a cellulose derivative, a polyvinyl acetate derivative or a (meth)acrylate copolymer.

5. The method as claimed in claim 1 wherein the film-forming coating agents are present in a mixing ratio of from 9:1 to 1:9 based on the total polymer content of the film coating.

6. The method as claimed in claim 1 wherein two or more two-fluid nozzles or one or more three-fluid nozzles are employed as spray device.

7. The method as claimed in claim 1 wherein the spray application takes place in a drum coater, a coating pan, a fluidized bed apparatus or a spray sifter.

8. The method as claimed in claim 7, wherein the spray application takes place by means of spray devices as fixed installation.

9. A pharmaceutical form, parts of pharmaceutical forms, food supplements or parts thereof produced by a method as claimed in claim 1.

10. A pharmaceutical form or parts of pharmaceutical forms as claimed in claim 9, wherein an active ingredient from the class of analgesics, antiallergics, antiarrhythmics, antibiotics, chemotherapeutics, antidiabetics, antidotes, antiepileptics, antihypertensives, antihypotensives, anticoagulants, antimycotics, antiinflammatory agents, beta-receptor blockers, calcium antagonists and ACE inhibitors, bronchospasmolytics/antiasthmatics, cholinergics, corticoids (for internal use), diuretics, enzyme inhibitors, enzyme preparations and transport proteins, expectorants, geriatrics, gout remedies, influenza remedies, hormones and their inhibitors, hypnotics/sedatives, cardiac drugs, lipid-lowering agents, parathyroid hormones/calcium metabolism regulators, psychoactive drugs, sex hormones and their inhibitors, spasmolytics, sympatholytics, sympathomimetics, vitamins, wound-treatment agents, cytostatics, nucleic acids, proteins or peptides, is present.

11. A pharmaceutical form or parts of pharmaceutical forms as claimed in claim 9 wherein the pharmaceutical forms are multiparticulate.

12. A drum coater, coating pan, fluidized bed apparatus or spray sifter suitable for carrying out a method as claimed in claim 1, comprising one or more three-fluid nozzles as spray device.

13. A method as claimed in claim 1 comprising utilizing one or more spray devices.
